(19) United States
(12) Patent Application Publication
(10) Pub. No.: US 2020/0340986 A1
(43) Pub. Date: Oct. 29, 2020

(54) Test Strip to Identify Insect and Arachnid Ectoparasites

(71) Applicants: Robert S. GOLD, Newburgh, IN (US); PARASITE ID, LLC, Newburgh, IN (US)

(72) Inventor: Robert S. Gold, Newburgh, IN (US)

(21) Appl. No.: 16/760,730

(22) PCT Filed: Oct. 31, 2018
(86) PCT No.: PCT/US18/58398

§ 371 Date: Apr. 30, 2020

Related U.S. Application Data
(60) Provisional application No. 62/579,650, filed on Oct. 31, 2017.

(51) Int. Cl. G01N 33/543; C07K 16/18; G01N 21/78; G01N 33/68

(52) U.S. Cl. CPC G01N 33/54366; C07K 16/18; G01N 2333/43552; G01N 33/6803; G01N 21/78

(57) ABSTRACT

The a test strip is provided for detecting the presence of insect and/or arachnid ectoparasites and/or their eggs.

### RELATED APPLICATIONS

This application claims the benefit of U.S. Provisional Application No. 62/579,650 titled “Test strip to identify lice,” to Robert Steven Gold, filed Oct. 31, 2017, the entire disclosure of which is expressly incorporated by reference herein.

### BACKGROUND AND SUMMARY OF THE DISCLOSURE

A lateral flow test strip for a immunoassay containing specific proteins to insect and/or arachnid ectoparasites and/or their eggs is described. There is a need for a detection method for diagnosis of lice infestations.

Several proteins unique only to the head louse and/or nits have been identified using data from the Louse Genome project. An immunochemical reagent-coated lateral flow test strip is used to determine if a subject has lice. The user uses a comb to brush a subject's hair, inserts the comb in a lysis solution, and places the lateral flow test strip disclosure in the solution contained lysed lice and nit proteins. A color change within the immunochemical reagent zone of the solid step test membrane of the lateral flow test strip indicates the presence of louse and/or nits.

The preferred test strip has a backing member and attached thereto a sample receiving end pad and at a distance therefrom a finishing absorbent end pad. A solid step test membrane is provided between the pads. The solid step membrane is positioned in parallel relationship to the backing at a distance therefrom so that the backing and the membrane limit between themselves an air gap which is open at its edges. The gap functions as a sheltered reaction chamber for the immunological reaction in at least one immunochemical reagent zone taking place as a liquid is made to flow through said membrane. The test strip is manufactured by lamination and it is suitable for diagnostic and/or environmental immunoassays.

### BRIEF DESCRIPTION OF THE DRAWINGS

The aforementioned aspects of this disclosure will grow to be appreciated at a greater level once references to the following accompanying illustrations are expounded upon.

FIG. 1A is a perspective view of a preferred embodiment of the test strip according to the present disclosure;

FIG. 1B shows an exploded view of the preferred embodiment according to FIG. 1A;

FIG. 2 shows a solid step test membrane to be used in the lateral flow test strip according to the present disclosure and the immunochemical reagent zones according to one of its embodiments; and

FIG. 3 shows a schematic cross section of another embodiment of the lateral flow test strip according to the present disclosure.

Equivalent reference components point to corresponding parts throughout the several views. Unless otherwise indicated, the components are shown proportional to each other. Wherein, the illustrations depicted are manifestations of the disclosure, and such illustrations shall in no way be interpreted as limiting the scope of the disclosure.

### DETAILED DESCRIPTION

The present disclosure concerns a lateral flow test strip 12 that provides a rapid assay for the detection of insect and arachnid ectoparasites. Insect and/or arachnid ectoparasite lateral flow test strip 12 of the present embodiment of the disclosure is specific for lice and/or nits since it detects a protein that is specific to the louse.

An assay can be performed by dipping lateral flow test strip 12 into a solution (not shown) containing lysed specimen samples and observing for a color change on test strip 12 after a period of time in an immunoassay. Immunoassays akin to the one in the present disclosure are based on the use of antibodies, biological reagents which bind to select analytes with high specificity.

The general principle behind the present disclosure involves lateral flow-immunochromatography (LF-IC) technology. Lateral flow tests using LF-IC technology are devices intended to detect the presence (or absence) of a target analyte in sample (matrix). Typically, these tests are used for medical diagnostics either for home testing, point of care testing, or laboratory use. To date, most LF-IC is intended to operate on a purely qualitative basis, using detectable labels to generate signals perceivable by the naked eye but exhibiting sensitivities lower than those associated with conventional immunological methods, particularly the widely used enzyme-linked immunosorbent assay (ELISA). However, it is possible to measure colorimetric signals from target proteins captured at the immunochemical reagent zone on a solid step test membrane, as demonstrated by the present disclosure and others using the lateral-flow strip-based concept. A widespread and well-known application is the home pregnancy test.

The technology is based on a series of capillary beds, such as pieces of porous paper, micro-structured polymer, or sintered polymer. Each of these elements has the capacity to transport fluid (e.g., lysate solution) spontaneously. A sample receiving end pad acts as a sponge and holds an excess of sample fluid. Once soaked, the fluid migrates to a conjugate pad in which the manufacturer has imbued an immunoconjugate comprised of dried bio-active particles in a salt-sugar matrix containing components to optimize chemical reactions between the target molecule (e.g., insect and arachnid ectoparasite proteins) and its chemical partner (e.g., anti-insect and arachnid ectoparasite proteins) that has been immobilized on the bio-active particle's surface. When the sample fluid dissolves the salt-sugar matrix, it releases the bio-active particles, and in one combined transport action, the sample and conjugate mix while flowing through the porous solid step test membrane. In this way, the target molecules bind to the bio-active particles while migrating further through the membrane. The solid step test membrane has one or more zones wherein an additional immunochemical reagent has been immobilized by the manufacturer. When the sample-conjugate mix reaches these zones, the third immunochemical reagent “capture” molecule binds the complex. When a sufficient amount of sample fluid passes through the immunochemical reagent zone, bound particles accumulate, and the immunochemical reagent zone changes color. There can be several test zones within each immunochemical reagent zone of the lateral flow test strips, each containing an immunochemical reagent specific to a particular protein target for specific colorimetric identification.

The analytical protocol of the present disclosure involves the capture of lice- and/or nit-specific proteins on antibodies bound to the test strip, forming an antibody-protein conjugate that can be detected by a colorimetric change on the lateral flow test strip to test for the presence of insect and arachnid ectoparasites, like lice and/or nits. Lice and/or nit samples are collected from a subject using a comb (not shown) used to brush a subject's hair. The comb is then submerged in a lysate solution containing sequence-grade Lys-C/Trypsin (Promega) to enzymatically digest the whole lice and/or nit samples.

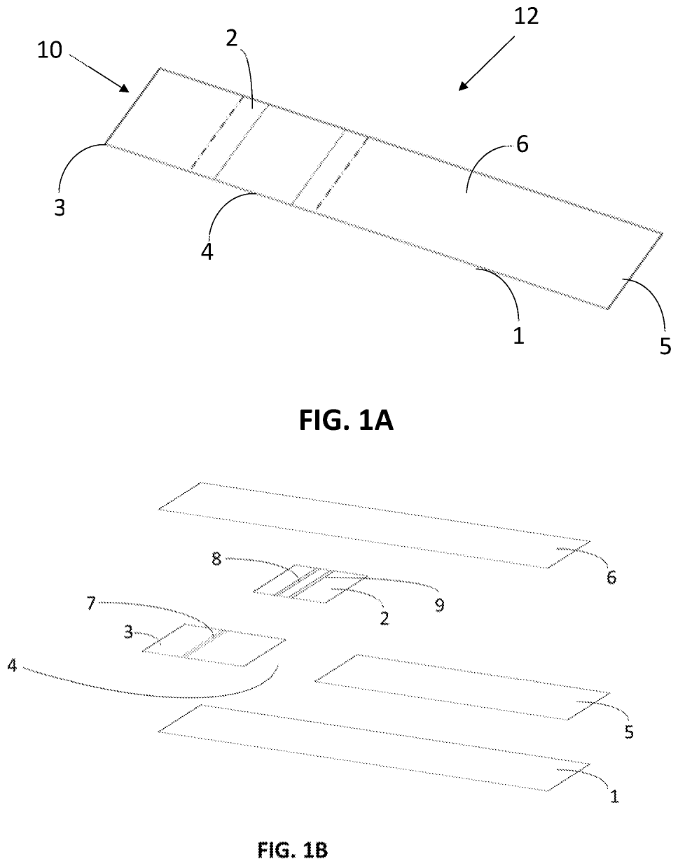

FIGS. 1A and 1B show a preferred embodiment lateral flow test strip 12 according to the present disclosure. A back side 10 of test strip 12, is formed by a backing member or bae 1 made of plastic or some other suitably stiff material. Both ends of backing member 1 are provided with relatively thin sample receiving end pad 3 and finishing absorbing pad 5. Pads 3, 5 are preferably made of a material that has a high absorbing capacity and is chemically inert. Sample receiving end pad 3 at the receiving end may be treated with various substances in order to increase its absorbing capacity and to increase the speed of liquid flow therein. An immunochemical reagent zone 7 in sample receiving end pad 3 contains a dried bio-active reagent.

Solid step test membrane 2, which is where the reaction to be detected will take place, is placed on and between sample receiving end pad 3 and finishing absorbing pad 5. The test is based on the lateral flow principle. Thus, immunological reactions occur on membrane 2, the chemical properties of which are chosen so as to make it possible to apply thereto immunological reagents and other substances needed in the reaction. Membrane 2 should be porous in a way suitable to create a liquid flow therein and to enable the substances needed for detection (e.g. metal colloid or other particles as well as soluble molecules) to be carried by the flow. A suitable material for the solid step test membrane 2 is polyvinylidene difluoride or nitrocellulose. Membrane 2 may alternatively be made of nylon or some other suitable material. If test membrane 2 is very thin, it is advantageous to attach it to a support of its own (not shown in Figs.). This will facilitate its treatment during the coating processes that precede test assembly. If detection of the reaction is to be viewed on the side having the membrane support, the support should be transparent.

The sample receiving end pad 3 of lateral flow test strip 10 is inserted into the protein-rich lysate solution (not shown) containing the digested lice and/or nit samples. In use, it is best to avoid dipping the zones of test membrane 2 into the sample liquid. The reactions may occur while the liquid front is proceeding along membrane 2. Consequently, the depth of dipping should be indicated to the user and most preferably a mark indicating the dipping level is marked on test strip 12 itself. One way of performing the test is to dip the sample receiving end of test strip 12 into a sample up to a marked line until sample receiving end pad 3 at the receiving end has absorbed a sufficient amount of liquid; thereafter test strip 10 can be removed from the sample and set aside until the visual indicator has appeared.

The louse- and/or nit-specific proteins bind to detectable label-conjugated antibodies specific to the identified louse- and/or nit-specific proteins disclosed in the present disclosure. The lice and/or nit protein-antibody complex migrates as the solution is made to flow through lateral flow test strip 10 via capillary action and binds a second nit- and/or louse-protein specific antibody located in the immunochemical reagent zone 7 of solid step test membrane 2 to form an antibody sandwich complex within the immunochemical reagent zone 7. The secondary binding results in a visible color change due to the optical properties of detectable labels bound to the antibodies in the sandwich complex on the lateral flow test strip, indicating the presence of lice or nits in the test sample solution.

Sample receiving end pad 3 and finishing absorbing pad 5 are separated by a gap 4 which is long enough to extend over the immunochemical reagent zone 7 in solid step test membrane 2, i.e. the area containing the reaction zone or zones. The zones at gap 4 comprise at least one specific antibody line 8, as well as any control line(s) 9. When test strip 12 is manufactured, solid step test membrane 2 is placed on top of sample receiving end pad 3 and finishing absorbing pad 5 in such a way that the exposed reactive test side of solid step test membrane 2 is facing gap 4. The contact area between solid step test membrane 2 and sample receiving end pad 3 and finishing absorbing pad 5, respectively, should be large enough to create and maintain a liquid flow.

The above arrangement results in a structure, wherein the reaction surface in solid step test membrane 2 is not in contact with any material. Instead, there is a gap 4 between solid step test membrane 2 and backing member 1, which gap can be described as a small chamber, open on both sides, where the liquid flow on solid step test membrane 2 is undisturbed. Sample receiving end pad 3 and finishing absorbing pad 5 are advantageously approximately 0.5-3 mm thick so that gap 4 will be narrow. Since gap 4 is narrow, the atmosphere in the gap 4 retains its humidity, and any drying caused by air draft will not disturb the flow of liquid.

A two-step procedure is employed to test for the presence of lice and nits using a lateral flow test strip, the procedure consisting of (i) initiation of sample flow to induce an antigen-antibody (or antigen-aptamer) reaction, and (ii) initiation of the flow of enzyme substrates for colorimetric signal generation.

First, the sample containing the digested lice and nit proteins applied to the sample receiving end pad 3 induces immunocomplex formation within the immunochemical reagent zone 7. In lateral flow assays, the interaction within the immunochemical reagent zone 7 is governed by capillary action, which can be rate limiting. An embodiment of the present disclosure proposes to increase the interactions and the number of targets by applying a magnetic field to ensure that a higher number of the lice- and/or nit-captured gold-coated iron nanoparticle (AuFe) probes interact within the immunochemical reagent zone 7 to enhance the signal. Signal enhancement is possible by using enzyme tracers and magnetic focusing of the target proteins to increase the number of targets in the immunochemical reagent zone 7 using AuFe probes.

As exemplified by ELISA, enzymes are another class of detectable labels that can be utilized in LF-IC. An enzyme tracer can catalyze a reaction to generate a strong signal measurable with comparatively simple detectors (e.g., colorimetry, chemiluminometry, or electrochemistry). Further, signal enhancement is possible by using enzyme tracers and magnetic focusing of the target proteins to increase the number of targets in the immunochemical reagent zone 7 using gold-coated iron nanoparticle (AuFe) probes.

Second, two horizontally arranged pads are placed on each lateral side of the signal generation pad, and the substrate (Tetramethylblue, TMB) is then added onto the supply pad to initiate enzymatic signal generation (horizontal flow) by reacting with the enzyme (Horseradish peroxidase, HRP). At the sites of complex formation, colorimetric signals can be produced by supplying the enzyme substrates at the time of signal generation almost instantaneously, a benefit of the cross-flow system, to result in a signal that is 102-104 folds higher than the conventional system. A lateral flow test strip may consist of different types of membranes to expedite the reaction (preferably less than 15 minutes) to further enable the in-situ separation of unreacted components via a one-step analysis.

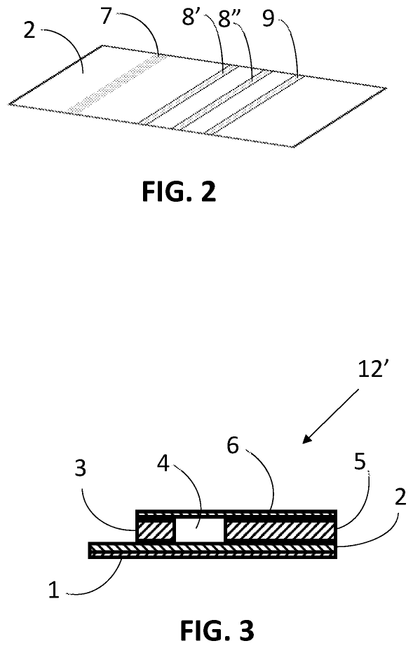

FIG. 2 presents another embodiment of the immunochemical reagent zone 7 where all the zones have been dispensed on solid step test membrane 2. Reference numeral 7 indicates a zone wherein bio-active particles used as label have been dried on a layer of a dried salt-sugar matrix; zones 8′ and 8″ contain immobilized immunochemical reagents specific to different analytes; and zone 9 has control immunochemical reagents attached to it.

The number and position of test and control zones on solid step test membrane 2 can vary depending on the analyte(s) to be tested. The same membrane may contain several immunological reagents for detecting different analytes in the same sample or correspondingly, for detecting different concentrations of the same analyte. The strip may also include several test membranes. In such a case, there must be an uninterrupted flow between them, which may be achieved, for instance, by placing the membranes at least partly on top of each other or by arranging a porous interconnecting membrane between them.

Using a test strip in accordance with the present disclosure, it is possible to perform a test, for example a louse protein test in solution, by placing the receiving end of strip 12 up to a certain level into the sample or an extract of the original sample. Liquid will be absorbed into sample receiving end pad 3 at the receiving end, where it will migrate further to solid step test membrane 2. Control zone 9 will become visible as soon as the liquid flow has reached the line. When the test is based on an immunometric principle, the test is positive if another, specific test line has also become colored.

Another backing, namely a housing 6 is advantageously attached on top of solid step membrane 2 and sample receiving end pad 3 and finishing absorbing pad 5. Housing 6 may have a transparent part preferably of the same size as the solid step test membrane 2 and made, for instance, of clear plastic. The housing protects solid step membrane 2 and sample receiving end pad 3 and finishing absorbing pad 5, keeps liquids from evaporating, and strengthens the structure. Because housing 6 is at least transparent over solid step test membrane 2, the colorimetric test result signal will be visible. Housing 6 may be painted in a suitable way or it may be provided with a tape or tapes (not shown) which facilitate interpretation of the test by covering the other parts of the strip except the area, where the test lines are formed. Housing 6 may also be made of nontransparent material with an opening or openings cut therein to enable the test result to be seen. Alternatively, backing member 1 will be transparent and the reaction can be observed through gap 4.

FIG. 3 presents an alternative embodiment of test strip 12′ according to the present disclosure, wherein backing member 1 carries the porous solid step test membrane 2. At a certain distance from a receiving end of the membrane there is an inert supporting sample receiving end pad 3. Another supporting finishing absorbing pad 5 is attached to the opposite end of the membrane-backing combination so that an empty space remains between sample receiving end pad 3 and finishing absorbing pad 5. On top of sample receiving end pad 3 and finishing absorbing pad 5, a housing 6 is attached to cover the pads in a way that results in the formation of a gap 4 which is open at its edges. Either backing member 1 or housing 6 or both are made of a transparent material over the immunochemical reagent zone 7.

In this embodiment, solid step test membrane 2 simultaneously acts as an absorbing pad, and liquid is absorbed directly into the membrane. Housing 6 and/or sample receiving end pad 3 may also have an extension equal to backing member 1, in which case liquid is absorbed into solid step test membrane 2 through its exposed edges.

To test the lice and/or nits using lateral flow test strips, lice and/or nit samples were collected in a laboratory setting, analyzed, characterized, and then placed on the test strip to determine if a colorimetric change occurred. In order to pick viable proteins for detection, lice/nit samples were placed in purified water for 5 minutes, and then the water was removed. Protein was precipitated using acetone before being dried. The samples were reduced and alkylated. All digestions were carried out in a Barocycler NEP2320 (Pressure Biosciences Inc.) at 50° C. under 20,000 psi for 1 hour. Digested samples were cleaned over C18 spin columns (Nest Group) and dried. Resulting pellets were resuspended in 97% purified H2O/3% ACN/0.1% formic acid (FA). Five μL of volume was used for nano LC-MS/MS analysis.

Using LC-MS/MS, the samples were run on a Dionex UltiMate 3000 RSLC Nano System coupled to the Q Exactive™ HF Hybrid Quadrupole-Orbitrap Mass Spectrometer (Thermo Scientific, Waltham, Mass.). Sample was first loaded onto a 300 μm i.d×5 mm C18 PepMap™ 100 trap column and washed 5 minutes with 98% purified water/2% acetonitrile (ACN)/0.1% formic acid (FA) at a flow rate of 5 μl/min. After 5 minutes, the trap column was switched in-line with a 75 μm×50 cm reversed phase Acclaim™ C18 PepMap™ 100 analytical column that was heated to 50° C. Peptides from the digestion were eluted from the columns using a mobile phase A of purified H2O/0.1% FA and a mobile phase B of 80% ACN/0.1% FA. With a flow rate of 0.3 μl/min, the method started at 2% B and reached 10% B in 5 minutes, 30% B in 80 minutes, 45% B in 91 minutes, and 100% B in 93 minutes. 100% B was held for 5 minutes before being brought to 2% B and held for 20 minutes. Sample was injected into the QE HF through the Nanospray Flex™ Ion Source fitted with an emitter tip (New Objective). The data acquisition was performed monitoring 20 precursor ions at 120,000 resolution and an injection time of 100 ms. Mascot Daemon v.2.5.1 (Matrix Science) was used for database searches against UniProt Pediculus humanus database.

All Pediculus humanus corporis protein sequences were downloaded from UniProtKB, a free and available database containing protein sequences and functional information for the protein sequences. The mass spectrometry data collected were compared against the downloaded lice proteins as well as the sequences within a database containing proteins unique to lice. Each protein contained in these databases has a unique identifier, called an accession number. Proteins identified in both databases were chosen as possible targets for detecting the presence of lice due to their abundance and the fact that they are unique to lice. Since the identified target proteins lacked functional information, other open source tools, like TargetP, which helps predict where the protein can be found, were also used. Searching the identified proteins in TargetP predicted that the proteins are part of a secretory pathway, which indicates their availability on the surface of lice.

The following protein sequences represent unique lice and nit protein identified in the samples using UniProt_PHC.

Lice Unique Protein: >PHUM604520|PHUM604520-RA
MADFKILLIAVLALFSLECQSAQVPKSATNLDLRLFVENSETVAKTLLSS
LNDALDKVKPNVDVMVEELSPEAKTLVKEAVKEGREKLAKEQERVNVVFD
TLNKAMKDLEPTKSCADKFSGPGKKWAHETEKKFVEAYEKVMKKHGKVLE
NLKVGGEKVLKNARQLFDEKVPKFVACWTPGKKDSKECVNKEVQSTLQEG
LKLVSDLTTLMQTADADLPPVMEDMLGKLAPVALGAMTGMTDLMNKFSDC
VANLK
Nits Unique Protein: >PHUM394730|PHUM394730-RA
MKPGVFILFIFDVLQFSQTEIRHRQQDKYATKQNYTTIPWSSSTTTYLNK
KNNNNLQKENYYLTTNVSKNDWTVTMPHVVSSKNGDDYDFRSITSQSSSR
SVKKTTTNHFSYVDTTDDDNKIVVNDDDDDDDDVDNDDVIKYYDSNDNRT
NYDDDDDRDFIGVKHETTREYSIATFKKINDEKITGKFVDDSPEEKFIFY
NESHLESSTQSHVSTNDGIFHRVKENVAVMKNKNRRSDDDDNNNTTSPSF
VGNNNKNFYFAQPLVALAESVDGIVDDDDNNNNNNNNIDDDCFENIVENF
SITDDIVSENILTDRNEAEKLTNVLARNKGDLDKMTDVMDSIMAEHDRIS
SISVVKLKKTDGSRISQTVYRKGSEILISRRSDRTFLLPVNNNNNNSWTK
IFKNCLTKFWTFGYYFFIENDTVR

The elements that are necessary to the lice and/or nit detection lateral flow test strip are the unique identified lice and/or nit proteins. Synthetic genes were created for each identified protein sequence. Then, the lice and nit recombinant proteins were expressed and purified. Characterized purified recombinant proteins were used to make polyclonal antibodies. Specifically, rabbits were immunized to generate the specific antibodies to lice and nit proteins. Purification of the target antibodies was obtained using peptide affinity columns.

Lateral flow test strips were developed using the antibodies created from unique proteins discovered from the lice and nits. It is obvious that test strips according to the present disclosure may be prepared one by one using pre-cut parts of the right size. However, owing to the test strip according to the disclosure having a laminated structure which is open at its edges, an industrial manufacturing method that is quick, automatic and cost effective has been developed. Thus, the test membrane 2 with its immunochemical reagent zone 7 may be pre-manufactured in a special production line and thereafter the test strip may be assembled in another production line by laminating the various components of the strip into a long test strip band.

The lateral flow test strips were used to test for the presence of lice and/or nits, and a colorimetric change was detected within the immunochemical reagent zone 7 on the lateral flow test strip. The test strip may, however, be adapted also for many other diagnostic tests where the presence or absence of some particular compound is detected in the sample. For example, the disclosure can be used to also test for other insects or arachnid ectoparasites such as other types of lice, scabies, or bed bugs, upon identification of unique proteins within these organisms and production of antibodies targeted to the unique proteins.

What is claimed is:

1. An insect and arachnid ectoparasite detection system, comprising:
a lateral flow strip, comprising:
a backing member;
a sample receiving end pad positioned on the backing member;
a finishing absorbent end pad located a distance downstream of the sample receiving end pad;
a conjugate pad inserted between the sample receiving end pad and the backing member and in liquid flow contact with the sample receiving end pad;
a solid step test membrane inserted between the conjugate pad and the backing member and in liquid flow contact with the conjugate pad and the finishing absorbent end pad; and
at least one immunochemical reagent zone positioned on the solid step test membrane and in a parallel relationship to the backing member.

2. An insect or arachnid ectoparasite detection system according to claim 1 wherein said receiving end pad and said finishing end pad are of a liquid absorbing material.

3. An insect or arachnid ectoparasite detection system according to claim 1, wherein a housing which has at least one transparent portion is positioned over the sample receiving end pad and the solid step test membrane, the at least one transparent portion being positioned over the at least one immunochemical reagent zone of the solid step test membrane.

4. An insect or arachnid ectoparasite detection system strip according to claim 3 wherein the solid step test membrane is made of a porous material.

5. An insect or arachnid ectoparasite detection system according claim 3 wherein the conjugate pad contains movable detectable particles coated with an immunochemical reagent, and wherein the at least one immunochemical reagent zone on the solid step test membrane contains at least one test zone containing immobilized immunochemical reagents and at least one control zone containing immobilized immunochemical reagents for indicating proper flow conditions.

6. An insect or arachnid ectoparasite detection system according to claim 5 wherein the conjugate pad contains different specific immunochemical reagents for more than one insect or arachnid ectoparasite analyte to be detected and/or different concentrations of one or more specific immunochemical reagents.

7. An insect or arachnid ectoparasite detection system according to claim 5 wherein the conjugate pad contains detectable particles coated with antibodies comprising:
a. Putative uncharacterized Pediculus humanus corporis protein >PHUM604520|PHUM604520-RA antibody; and
b. Putative uncharacterized Pediculus humanus corporis protein >PHUM394730|PHUM394730-RA antibody.

8. An insect or arachnid ectoparasite detection system according to claim 1, wherein the at least one immunochemical reagent zone comprises immobilized immunochemical reagents specific for one or more environmental analytes.

9. An insect or arachnid ectoparasite detection system according to claim 1, wherein the at least one immunochemical reagent zone comprises immobilized immunochemical reagents specific for one or more diagnostic analytes.

10. An insect or arachnid ectoparasite detection system according to claim 4, wherein said porous material is polyvinylidene fluoride or polyvinylidene difluoride.

11. An insect or arachnid ectoparasite detection system according to claim 5, wherein the at least one immunochemical reagent zone comprises immobilized immunochemical reagents specific for one or more insect or arachnid ectoparasite analytes including antibodies comprising:
a. Putative uncharacterized Pediculus humanus corporis protein >PHUM604520|PHUM604520-RA antibody; and
b. Putative uncharacterized Pediculus humanus corporis protein >PHUM394730|PHUM394730-RA antibody.

12. An insect or arachnid ectoparasite detection system according to claim 5, wherein chemical interaction of movable detectable particles coated with an immunochemical reagent with at least one test zone containing immobilized immunochemical reagents results in development of visible color to indicate the presence of an insect or arachnid ectoparasite analyte.

13. An insect or arachnid ectoparasite detection system comprising
a base,
at least one of a lice and nit protein-antibodies supported by the base, and,
detectable labels associated with the antibodies.

14. The detection system of claim 13, wherein the at least one of the antibodies comprises:
a. Putative uncharacterized Pediculus humanus corporis protein >PHUM604520|PHUM604520-RA antibody; and
b. Putative uncharacterized Pediculus humanus corporis protein >PHUM394730|PHUM394730-RA antibody.

15. A method of detecting an insect or arachnid ectoparasite comprising the steps of
collecting at least one of lice and a nit,
creating a solution with a liquid and the at least one lice and nit,
exposing the solution to an antibody of a protein the at least one lice and nit, the protein antibody being a component of a detection system, and
detecting a visible change in the detection system to detect the presence of the protein of the at least one lice and nit.

16. The method of claim 15, wherein the protein is substantially unique to at least one of a lice and nit.

17. The method of claim 15, wherein the at least one lice and nit are digested in the solution creating step.

18. The method of claim 15, wherein the at least one lice and nit are collected from a person's hair.
